(19) United States
(12) Patent Application Publication
(10) Pub. No.: US 2013/0030258 A1
(43) Pub. Date: Jan. 31, 2013

(54) COPD EXACERBATION PREDICTION SYSTEM AND METHOD

(71) Applicants: Amy Oi Mee Cheung, Eindhoven (NL); Maryam Atakhorrami, Cambridge (GB); Geert Guy Georges Morren, Vissenaken (DE)

(72) Inventors: Amy Oi Mee Cheung, Eindhoven (NL); Maryam Atakhorrami, Cambridge (GB); Geert Guy Georges Morren, Vissenaken (DE)

(73) Assignee: KONINKLIJKE PHILIPS ELECTRONICS N.V., EINDHOVEN (NL)

(21) Appl. No.: 13/512,962

(22) PCT Filed: Nov. 17, 2010
(86) PCT No.: PCT/IB10/55220

§ 371 Date: May 31, 2012

Related U.S. Application Data
(60) Provisional application No. 61/288,271, filed on Dec. 19, 2009.

(51) Int. Cl. A61B 5/0205; A61B 5/11; A61B 5/08

(52) U.S. Cl. 600/301

(57) ABSTRACT

A computer-implemented method for predicting an onset of an exacerbation in a COPD patient is provided. The method includes measuring physical activity of the patient over a period of time to gather physical activity data; measuring a respiration characteristic of the patient over the period of time to gather respiration data; and executing, on one or more computer processors, one or more computer program modules to detect the onset of the exacerbation based on predetermined criteria, wherein the predetermined criteria comprises a comparison of a change in the respiration data with a change in the physical activity data.

This patent application claims the priority benefit under 35 U.S.C. §119(e) of U.S. Provisional Application No. 61/288,271 filed on Dec. 19, 2009, the contents of which are herein incorporated by reference.

The present invention relates to a method and a system for predicting an onset of an exacerbation in patients suffering from COPD.

Chronic Obstructive Pulmonary Disease (COPD) is a respiratory disease that is characterized by inflammation of the airways. COPD is characterized by an airflow limitation that is not fully reversible. The airflow limitation is both progressive and associated with an abnormal inflammatory response of the lungs to noxious particles or gases. Symptoms of COPD may include coughing, wheezing and the production of mucus, and the degree of severity may, in part, be viewed in terms of the volume and color of secretions.

Exacerbations are the worsening of COPD symptoms. The exacerbations may be associated with a variable degree of physiological deterioration. The exacerbations may be measured as a decrease in Forced Expiratory Volume measured over one second (FEV₁). The exacerbations may be characterized by increased coughing, dyspnea (i.e., shortness of breath) and production of sputum. The major symptom of an exacerbation is the worsening of dyspnea (i.e., shortness of breath) while the main reaction is a lack of energy, which in turn may translate to a reduction in physical activity levels.

The exacerbations are normally caused by viral or bacterial infections and often may lead to hospitalization of the COPD patients. The frequency of exacerbations increases during the winter months due to cold stresses on the patient's body. This may be due to a combination of a) the cooling of facial skin and airways, resulting in bronchoconstriction, and b) the thermoregulatory system becoming less effective with age, thus making COPD patients more susceptible for respiratory infections. The exacerbations not only limit the performance of daily activities, but also significantly decrease the health related quality of life of COPD patients. A high frequency of exacerbations is linked to a poor prognosis for survival. Also, the exacerbations often may result in hospitalization, which is the main determinant of the overall healthcare expenditure for COPD patients.

Because of the damage done when an exacerbation takes place it is desirable to predict the likely onset of an exacerbation and initiate treatments which either prevent the exacerbation occurring and/or treat the symptoms at an early stage thereby reducing the damage caused by the exacerbation. Moreover, reducing and most importantly preventing exacerbations may help COPD patients lead an improved quality of life and may lower the healthcare costs for COPD patients.

Questionnaires are used in clinical trials to confirm the occurrence of an exacerbation. The questionnaires used to confirm the exacerbations may include weekly questionnaires. The weekly questionnaires are designed to be more comprehensive, however tracking of symptoms is less frequent and thus there is a delay of identifying an exacerbation using these weekly questionnaires. Typically, a general practitioner or a hospital physician will confirm if a patient has an exacerbation.

One aspect of the present invention provides a computer-implemented method for predicting an onset of an exacerbation in a COPD patient. The method includes measuring physical activity of the patient over a period of time to gather physical activity data; measuring a respiration characteristic of the patient over the period of time to gather respiration data; and executing, on one or more computer processors, one or more computer program modules to detect the onset of the exacerbation based on predetermined criteria. The predetermined criteria includes a comparison of a change in the respiration data with a change in the physical activity data.

Another aspect of the present invention provides a system for predicting an onset of an exacerbation in a COPD patient. The system includes at least one sensor, and at least one processor. The sensor is configured a) to measure physical activity of the patient over a period of time to gather physical activity data, and b) to measure a respiration characteristic of the patient over the period of time to gather respiration data. The processor is configured to detect the onset of the exacerbation based on predetermined criteria. The predetermined criteria includes a comparison of a change in the respiration data with a change in the physical activity data.

Another aspect of the present invention provides a computer-implemented method for predicting an onset of an exacerbation in a COPD patient. The method includes measuring physical activity of the patient over a period of time to gather physical activity data; measuring a respiration characteristic of the patient over the period of time to gather respiration data; measuring a heart rate of the patient over the period of time to gather heart rate data; and executing, on one or more computer processors, one or more computer program modules to detect the onset of the exacerbation based on predetermined criteria. The predetermined criteria includes a comparison of a change in the respiration data and a change in the heart rate with a change in the physical activity data.

Another aspect of the present invention provides a system for predicting an onset of an exacerbation in a COPD patient. The system includes at least one sensor, and at least one processor processing device. The sensor is configured to a) measure physical activity of the patient over a period of time to gather physical activity data; b) measure a respiration characteristic of the patient over the period of time to gather respiration data; and c) measure a heart rate of the patient over the period of time to gather heart rate data. The processor is configured to detect the onset of the exacerbation based on predetermined criteria. The predetermined criteria includes a comparison of a change in the respiration data and a change in the heart rate data with a change in the physical activity data.

Another aspect of the present invention provides a system for predicting an onset of an exacerbation in a COPD patient. The system includes means for measuring physical activity of the patient over a period of time to gather physical activity data; means for measuring a respiration characteristic of the patient over the period of time to gather respiration data; and means for detecting the onset of the exacerbation based on predetermined criteria, wherein the predetermined criteria comprises a comparison of a change in the respiration data with a change in the physical activity data.

Another aspect of the present invention provides a system for predicting an onset of an exacerbation in a COPD patient. The system includes means for measuring physical activity of the patient over a period of time to gather physical activity data; means for measuring a respiration characteristic of the patient over the period of time to gather respiration data; means for measuring a heart rate of the patient over the period of time to gather heart rate data; and means for detecting the onset of the exacerbation based on predetermined criteria, wherein the predetermined criteria comprises a comparison of a change in the respiration data and a change in the heart rate data with a change in the physical activity data.

These and other aspects of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. It shall also be appreciated that the features of one embodiment disclosed herein may be used in other embodiments disclosed herein. As used in the specification and in the claims, the singular form of “a”, “an”, and “the” include plural referents unless the context clearly dictates otherwise.

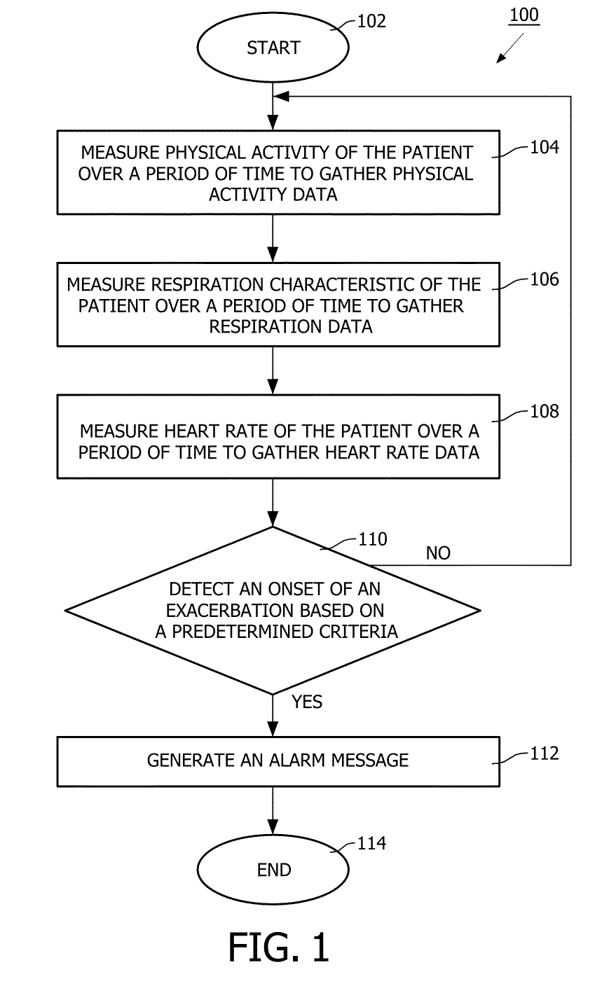

FIG. 1 is a flow chart illustrating a method for predicting an onset of an exacerbation in a patient in accordance with an embodiment of the present invention;

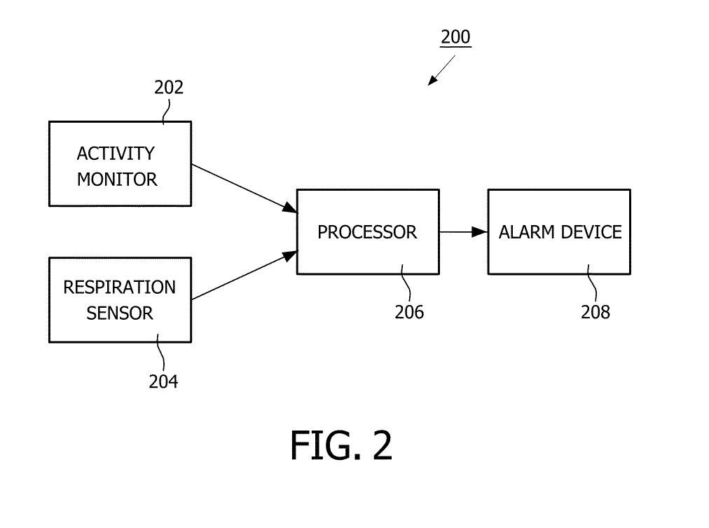

FIG. 2 shows a system for predicting the onset of an exacerbation in a patient in accordance with an embodiment of the present invention;

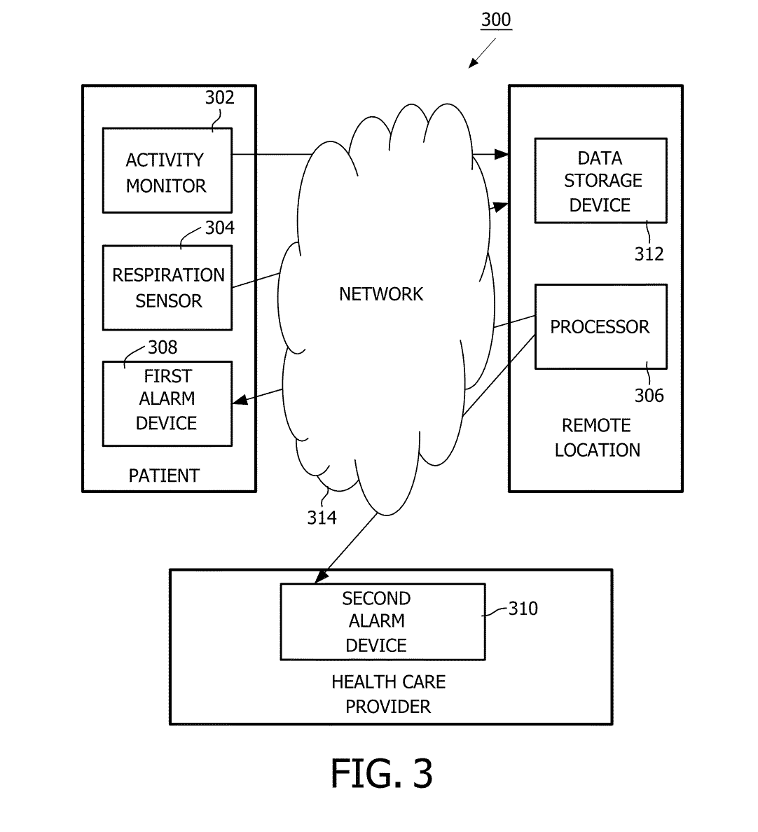

FIG. 3 shows a system for predicting the onset of the exacerbation in a patient in accordance with another embodiment of the present invention;

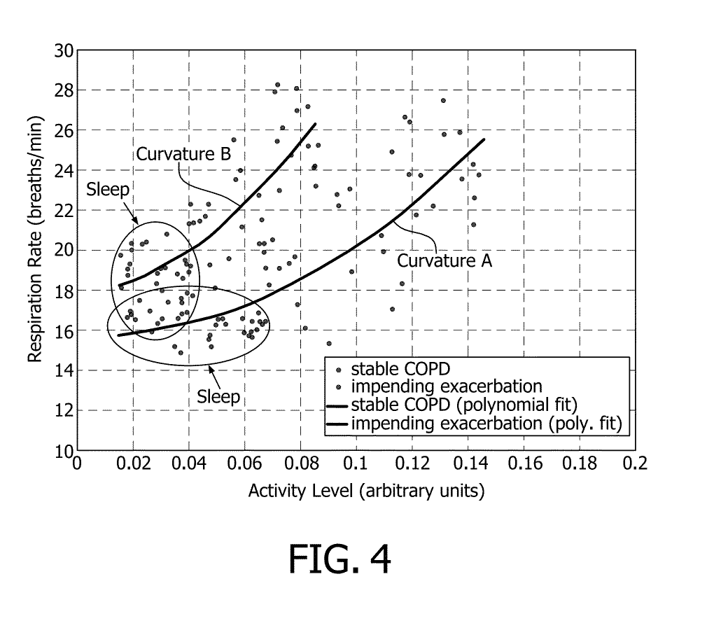

FIG. 4 shows a graphical representation providing an exemplary correlation between the physical activity and the respiration characteristic (e.g., respiration rate) in accordance with an embodiment of the present invention;

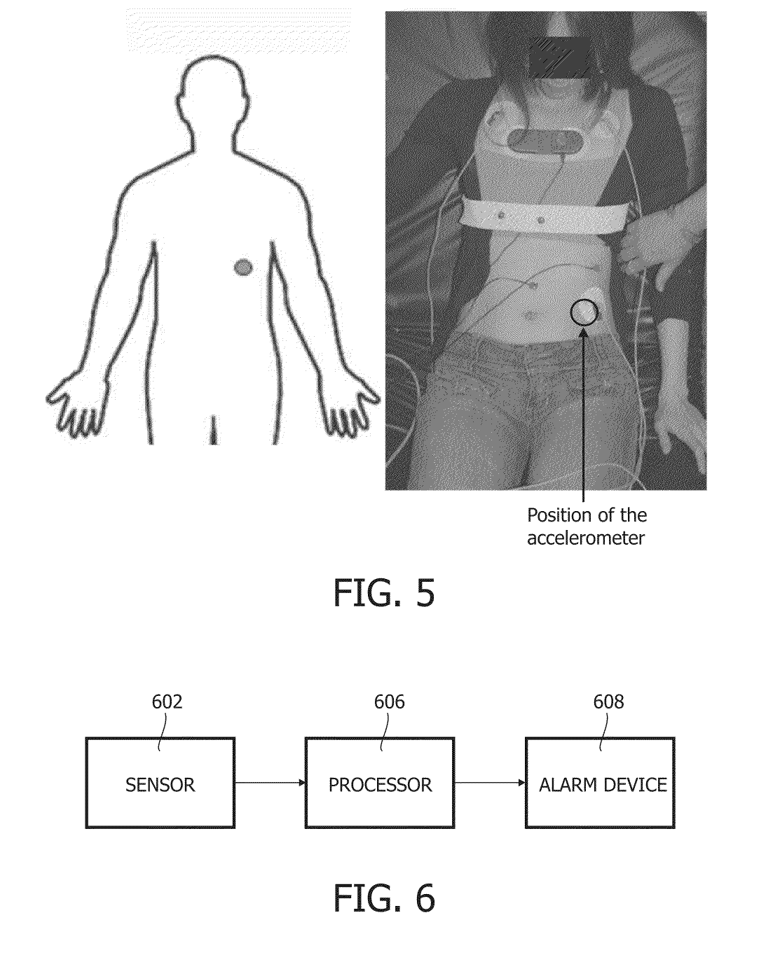

FIG. 5 shows the positioning of the accelerometer in accordance with another embodiment of the present invention; and

FIG. 6 shows a system using a single sensor for predicting the onset of an exacerbation in a patient in accordance with another embodiment of the present invention.

FIG. 1 is a flow chart illustrating a computer implemented method 100 for predicting an onset of an exacerbation in a COPD patient in accordance with an embodiment of the present invention. Method 100 is implemented in a computer system comprising one or more processors 206 (as shown in and explained with respect to FIG. 2), 306 (as shown in and explained with respect to FIG. 3) or 606 (as shown in and explained with respect to FIG. 6) configured to execute one or more computer programs modules. In one embodiment, the processor 206 (as shown in and explained with respect to FIG. 2), 306 (as shown in and explained with respect to FIG. 3) or 606 (as shown in and explained with respect to FIG. 6), each can comprise either one or a plurality of processors therein.

Method 100 begins at procedure 102. At procedure 104, a physical activity of the patient is measured over a period of time to gather physical activity data. The physical activity of the patient is measured over a period of time using an activity monitor, such as sensor 202 (as shown in and explained with respect to FIG. 2), sensor 302 (as shown in and explained with respect to FIG. 3) or sensor 602 (as shown in and explained with respect to FIG. 6). The period of time may include a day, a week, a month, or any other desired time period.

At procedure 106, a respiration characteristic of the patient is measured over the period of time to gather respiration data. The respiration characteristic of the patient may include a respiration rate or a respiration pattern. The respiration rate of the patient is measured over the period of time using a respiration sensor, such as sensor 204 (as shown in and explained with respect to FIG. 2), sensor 304 (as shown in and explained with respect to FIG. 3) or sensor 602 (as shown in and explained with respect to FIG. 6). The respiration rate is generally representative of number of breaths taken by a patient per minute.

At procedure 108, a heart rate of the patient is measured over the period of time to gather heart rate data. The heart rate of the patient is measured over the period of time using a heart rate sensor, such as sensor 602 (as shown in and explained with respect to FIG. 6).

In one embodiment, each of the physical activity, the respiration characteristic, and the heart rate of the patient may be measured (i.e., over the period of time) using separate sensors. In another embodiment, as shown in FIG. 6, a single sensor, such as the sensor 602, may be used measure the physical activity, the respiration characteristic, and the heart rate of the patient (i.e., over the period of time).

At procedure 110, a processor 206 (as shown in and explained with respect to FIG. 2), 306 (as shown in and explained with respect to FIG. 3) or 606 (as shown in and explained with respect to FIG. 6) is configured to detect the onset of the exacerbation based on predetermined criteria.

In one embodiment, as explained with respect to FIGS. 2 and 3, the predetermined criteria includes a comparison of a change in the respiration data with a change in the physical activity data over the period of time. The change in the respiration data indicates an increase in the respiration rate, and the change in the physical activity data indicates a decrease in the physical activity.

In another embodiment, as explained with respect to FIG. 6, the predetermined criteria includes a comparison of a change in the respiration data and a change in heart rate data with a change in the physical activity data over the period of time. The change in the respiration data indicates an increase in the respiration rate, the change in the heart rate data indicates an increase in the heart rate data and the change in the physical activity data indicates a decrease in the physical activity.

The respiration characteristic (e.g., respiration rate patterns) may provide an indication of the worsening of dyspnea (i.e., shortness of breath) as an increase in dyspnea is often followed by a rapid respiration rate due to increased difficulty in breathing. In one embodiment, method 100 is configured to monitor and analyze the trends in the physical activity data and the trends in the respiration rate of a patient to detect a decrease in activity level in combination with an increase in respiration rate for predicting the onset of an exacerbation. In other words, an increase in respiration rate over time together in combination with a decrease in activity levels may indicate worsening of dyspnea and lack of activity, both of which are strong predicators for the onset of an exacerbation.

In another embodiment, method 100 is configured to monitor and analyze the trends in the physical activity data and the trends in the respiration rate of a patient to detect an increase in respiration rate with a constant activity level or a decrease in activity level for predicting the onset of an exacerbation. In other words, an increase in respiration rate over time together with a constant activity level or a decrease in activity level may indicate worsening of dyspnea, which is a strong predicator for the onset of an exacerbation.

In another embodiment, method 100 is configured detect an increase in the respiration rate from a baseline respiration rate value with a constant activity level or a decrease in activity level from a baseline activity level value for predicting the onset of an exacerbation. In one embodiment, the baseline respiration rate value is a respiration rate value that is measured for low, moderate and high activity levels.

In another embodiment, method 100 is configured to monitor and analyze the trends in the physical activity data, the trends in the heart rate data, and the trends in the respiration rate of a patient to detect a decrease in the physical activity in combination with an increase in the respiration rate and the heart rate for predicting the onset of an exacerbation. In other words, an increase in the respiration rate and the heart rate over time together in combination with a decrease in the physical activity may indicate worsening of dyspnea and lack of activity, both of which are strong predicators for the onset of an exacerbation.

In another embodiment, method 100 is configured to monitor and analyze the trends in the physical activity data, the trends in the heart rate data, and the trends in the respiration rate of a patient to detect an increase in the respiration rate and the heart rate with a constant activity level for predicting the onset of an exacerbation. In other words, an increase in the respiration rate and the heart rate over time with a constant activity level or a decrease in activity level may indicate worsening of dyspnea, which is a strong predicator, for the onset of an exacerbation.

In another embodiment, method 100 is configured detect an increase in the respiration rate from a baseline respiration rate value and an increase in the heart rate from a baseline heart rate value with a constant activity level or a decrease in activity level from a baseline activity level for predicting the onset of an exacerbation. In one embodiment, as noted above, the baseline respiration rate value is a respiration rate value that is measured for low, moderate and high activity levels. In one embodiment, the baseline heart rate value is a heart rate value that is measured for low, moderate and high activity levels.

When the predetermined criteria is satisfied, then method 100 proceeds to procedure 112. If the predetermined criteria is not satisfied, then method 100 returns to procedure 104 where measuring of the physical activity of the patient is continued to the gather physical activity data over the period of time.

At procedure 112, an alarm indication or a warning may be generated by an alarm device, such as alarm device 208 (as shown in FIG. 2), alarm devices 308 and 310 (as shown in FIG. 3), or alarm device 608 (as shown in FIG. 6). The alarm indication may be generated to indicate that the onset of the exacerbations is detected. The alarm indication generated at procedure 112 may be then transmitted to a patient (as shown in system 200 of FIG. 2) and/or a healthcare provider (as shown in system 300 of FIG. 3). The alarm indication generated may alert the patient to take appropriate action, for example, take medication or intervention steps. In one embodiment, intervention steps may include pulmonary rehabilitation (includes smoking cessation). Method 100 ends at procedure 114.

In one embodiment, procedures 102-114 can be performed by one or more computer program modules that can be executed by one or more processors 206 (as shown in and explained with respect to FIG. 2), 306 (as shown in and explained with respect to FIG. 3) or 606 (as shown in and explained with respect to FIG. 6).

System 200 for predicting the onset of an exacerbation in a patient in accordance with an embodiment of the present invention is shown in FIG. 2. In one embodiment, system 200 of the present invention may be used by patients in the home environment of the patient.

System 200 may include the activity monitor 202, the respiration sensor 204, the processor 206, and the alarm device 208. In one embodiment, based on the obtained measurements (i.e., the monitored respiration rate from respiration sensor 204, and/or the monitored activity level from activity monitor 202), a score card is used to classify the patient into either a safe category, at risk category or action required category.

In one embodiment, processor 206 can comprise either one or a plurality of processors therein. In one embodiment, processor 206 can be a part of or forming a computer system.

Activity monitor 202 is configured to detect body movements of the patient such that a signal from the activity monitor is correlated to the level of a patient's physical activity. In one embodiment, activity monitor 202 may include an accelerometer. In one embodiment, the accelerometer may be a three-axis accelerometer. Such an accelerometer may include a sensing element that is configured to determine acceleration data in at least three axes. For example, in one embodiment, the three-axis accelerometer may be a three-axis accelerometer (i.e., manufacturer part number: LIS3L02AQ) available from STMicroelectronics.

In one embodiment, the output of the accelerometer may be represented in arbitrary acceleration units (AAU) per minute. The AAU can be related to total energy expenditure (TEE), activity-related energy expenditure (AEE) and physical activity level (PAL).

In another embodiment, activity monitor 202 may be a piezoelectric sensor. The piezoelectric sensor may include a piezoelectric element that is sensitive to body movements of the patients.

In one embodiment, activity monitor 202 may be positioned, for example, at the thorax of the patient or at the abdomen of the patient. In one embodiment, the activity monitor 202 may be a part of a wearable band (that may be worn on the wrist, waist, arm or any other portion of the patient's body for example) or may be part of wearable garment worn by the patient.

In one embodiment, the respiration rate sensor 204, which is configured to measure the respiration pattern of the patient, may include an accelerometer or a microphone. In one embodiment, the accelerometer may be a three-axis accelerometer. For example, in one embodiment, the three-axis accelerometer may be a three-axis accelerometer available from STMicroelectronics.

In one embodiment, a microphone is constructed and arranged to receive sound of inspiration of the patient in order to determine the respiration rate of the patient. In one embodiment, the respiration rate sensor 204 may be a Respiband™ available from Ambulatory Monitoring, Inc. of Ardsley, N.Y. In one embodiment, Respiband™ measures the respiration rate using inductance.

In one embodiment, the respiration rate sensor may include a chest band and a microphone as described in U.S. Pat. No. 6,159,147, the contents of which are hereby incorporated by reference. In such an embodiment, the chest band may be placed around a patient's chest to measure the patient's respiration rate, for example. Sensors on the chest band may measure movement of the patient's chest. Data from sensors on the chest band is input into a strain gauge and subsequently amplified by an amplifier.

Processor 206 is configured to a) receive the physical activity data from the activity monitor 202, b) receive the respiration data from respiration monitor 204, and c) analyze the physical activity data and the respiration data to detect the onset of exacerbations in the patient based on the predetermined criteria. As noted above, the predetermined criteria includes a comparison of a change in the respiration data with a change in the physical activity data over the period of time. The change in the respiration data indicates an increase in the respiration rate, and the change in the physical activity data indicates a decrease in the physical activity.

In one embodiment, an increase in the respiration rate is determined by comparing the patient's current respiration rate to the patient's prior respiration rate (e.g., a period of time ago). As noted above, the period of time may include a day, a week, a month, or any other desired time period.

In one embodiment, an increase in the respiration rate is determined by comparing the patient's current respiration rate to the baseline respiration rate. In one embodiment, as noted above, the baseline respiration rate is measured for low, moderate and high activity levels to provide a reference.

In another embodiment, an increase in the respiration rate is determined by comparing the patient's current respiration rate to the patient's average respiration rate. In one embodiment, the patient's average respiration rate is determined by calculating an average or a median of the respiration rate data taken over a past period of time.

In one embodiment, a decrease in the physical activity is determined by comparing the patient's current physical activity to the patient's physical activity a period of time ago. As noted above, the period of time may include a day, a week, a month, or any other desired time period.

In another embodiment, a decrease in the physical activity is determined by comparing the patient's current physical activity to the patient's average physical activity. In one embodiment, the patient's average physical activity is determined by calculating an average or a median of the physical activity data taken over a past period of time.

In one embodiment, the average respiration rate of the patient at rest is 12-18 breaths per minute. In one embodiment, an acute exacerbation is detected when the respiration rate of the patient at rest increases to greater than 25 breaths per minute.

In one embodiment, the average heart rate at rest is 60-100 beats per minute. In one embodiment, an acute exacerbation is detected when the heart rate increases to greater than 110 beats per minute.

In one embodiment, the processor 206 may include a data storage unit or memory (not shown) that is constructed and arranged to store the physical activity data and the respiration data over the period of time. The stored data may be used for further processing, for example, for trending, and/or display.

When the predetermined criteria is satisfied, processor 206 is configured to transmit a signal to alarm device 208 to generate the alarm indication. The alarm indication may be generated to indicate that the onset of the exacerbations is detected.

Alarm device 208 may include a sound producing device and/or a visual indicator. The sound producing device, if provided, is constructed and arranged to generate an audio alarm indication in response to the detection of the onset of the exacerbations in the patient. The visual indicator, if provided, is constructed and arranged to generate a visual alarm indication in response to the detection of the onset of the exacerbations in the patient.

In one embodiment, the sound producing device may include a speaker. In one embodiment, the audio alarm indication may include, but not limited, to a tone, a buzz, a beep, a sound (e.g., a horn or a chime), and/or a prerecorded voice message. In one embodiment, the audio alarm indication may include tones with changing frequency or volume. In one embodiment, the audio alarm indication may include customer configurable tones and alarms.

In one embodiment, the visual indicator may include one or more lights, lamps, light emitting diodes and/or liquid crystal displays. In an embodiment, the visual alarm indication may be generated by, for example, continuous, or flashing lights.

In one embodiment, alarm device 208 may be a part of the activity monitor and/or the respiration sensor. In one embodiment, the alarm device 208 may be positioned, for example, on the patient to provide the alarm indication to the patient. In another embodiment, alarm device 208 may be, for example, a stand alone device in the home environment of the patient to provide the alarm indication to the patient. In such an embodiment, alarm device 208 may be connected to processor 206 over the network. Also, in such an embodiment, alarm device 208 may be configured to transmit a signal or an alarm indication a to personal handheld device of the patient such as cellular phone, PDA, or other personal electronic device over a wired or a wireless network.

The alarm indication generated may alert the patient to take appropriate action, for example, take medication or intervention steps (e.g., smoking cessation). In one embodiment, it is also contemplated that system 200 may also be configured to transmit the alarm indication to the healthcare provider over the network (wired or wireless, for example) so that the healthcare provider, for example, may prescribe an appropriate medication or action that needs to be taken by the patient.

FIG. 3 shows system 300 for predicting the onset of an exacerbation in a patient in accordance with another embodiment of the present invention. System 300 includes an activity monitor 302, a respiration sensor 304, a processor 306, a data storage device 312, a first alarm device 308, and a second alarm device 310. System 300 is similar to system 200 described in the FIG. 2, except for the following aspects.

In one embodiment, processor 306 can comprise either one or a plurality of processors therein. In one embodiment, processor 306 can be a part of or forming a computer system.

Activity monitor 302 and respiration sensor 304 may include a transmission unit (not shown) that is configured to transmit the physical activity data and the respiration data to data storage device 312 located at a remote location via network 314. Network 314 may include a wired or a wireless connection, for example.

In one embodiment, the physical activity data and the respiration data stored in the data storage unit may be used for further processing, for example, for trending, and/or display. In such an embodiment, the physical activity data and the respiration data stored in the data storage unit may be downloaded automatically (e.g., at periodic intervals) or on command and presented to the healthcare provider to provide a trend of the physical activity data and the respiration data of the patient over the period of time. In such an embodiment, system 300 may include a user interface, which is in communication with processor 306. The user interface is configured to transmit (and display) output of the system 300.

Processor 306 is configured to a) receive the physical activity data from the data storage device 312, b) receive the respiration data from data storage device 312, and c) analyze the physical activity data and the respiration data to detect the onset of exacerbations in the patient based on the predetermined criteria. As noted above, the predetermined criteria includes a comparison of a change in the respiration data with a change in the physical activity data over the period of time. The change in the respiration data indicates an increase in the respiration rate, and the change in the physical activity data indicates a decrease in the physical activity.

In the illustrated embodiment, data storage device 312 and processing unit 306 are located at a remote location. In another embodiment, it is contemplated that the processor 306 and data storage device 312 of system 300 may be located at the healthcare provider's location rather than at the remote location.

When the predetermined criteria is satisfied, processor 306 transmits a signal over the network 314 to first alarm device 308 located in the home environment of the patient and/or to the second alarm device 310 located at the healthcare provider's location. First and second alarm devices 308 and 310 are configured to generate the alarm indication to indicate that the onset of the exacerbations is detected.

The alarm indication generated by first alarm device 308 may alert the patient to take appropriate action, for example, to take appropriate medication or intervention steps (e.g., smoking cessation). In addition, the alarm indication generated by second alarm device 310 may alert the healthcare provider to take appropriate action, for example, to provide appropriate medication or intervention steps.

FIG. 4 shows a graphical representation providing an exemplary correlation between the physical activity and the respiration characteristic (e.g., respiration rate) in accordance with an embodiment of the present invention. Such correlations may be used by processors 206, 306, or 606 to detect the onset of an exacerbation.

The exemplary correlation between the physical activity and the respiration characteristic (e.g., respiration rate) is taken, for example, over a course of day. The graph illustrates the physical activity, expressed in arbitrary units, on a horizontal x-axis. On a vertical y-axis, the graph illustrates the respiration rate, expressed in breaths/min.

The graphical representation includes the physical activity data and the respiration data for a stable patient, and the physical activity data and the respiration data for a patient with an impending exacerbation. Curvature A is obtained from a polynomial fit to the physical activity data and the respiration data for a stable patient, and curvature B is obtained from a polynomial fit to the physical activity data and the respiration data for a patient with an impending exacerbation. A polynomial fit function (i.e., generally know in the art) is used to obtain the curvatures A and B. Referring to the curvature B, it may be seen that the physical activity level is decreased and the respiration rate is increased during the early phase of an exacerbation.

FIG. 6 shows a system 600 that uses a single sensor for predicting the onset of an exacerbation in a patient in accordance with another embodiment of the present invention. In one embodiment, processor 606 of system 600 can comprise either one or a plurality of processors therein. In one embodiment, processor 606 can be a part of or forming a computer system.

System 600 is configured to predict an onset of an exacerbation in a patient by analyzing the objectively assessed physical activity, respiration characteristic and heart rate over a period of time (e.g., the course of day), and the correlations between these physiological parameters. In one embodiment, the objective assessment is done using an accelerometer (or one of the other sensors described above).

As shown above, the graphical representation in FIG. 4 provides an exemplary correlation between the physical activity and the respiration characteristic (e.g., respiration rate). In one embodiment, the data (as shown in FIG. 4) may be analyzed in several ways to detect an exacerbation. In one embodiment, the correlations between respiration rate and activity level (as shown in FIG. 4) are explicitly analyzed. In other words, the correlations between respiration rate and activity level would correspond to the slope of the curves in FIG. 4 (that are eventually restricted to a predefined range of activity levels). It is contemplated that correlation analyses similar to that shown in FIG. 4 may be made between the heart rate and the physical activity, or the respiration rate and the heart rate. Such correlations may be used by the processor 606 to detect the onset of an exacerbation.

In one embodiment, other parameters may enable detection of an exacerbation (i.e., besides the correlations discussed above). These parameters may include resting heart rate (HR) or respiration rate (RR) that are measured during a period of low activity such as, for example, sleep; and/or the median/average/maximum activity level during the day-time.

System 600 may include a sensor 602, a processor 606, an alarm device 608. In one embodiment, sensor 602 may be an accelerometer. In one embodiment, the accelerometer may be a three-axis accelerometer. Such an accelerometer may include a sensing element that is configured to determine acceleration data in at least three axes. For example, in one embodiment, the three-axis accelerometer may be a three-axis accelerometer (i.e., manufacturer part number: LIS3L02AQ) available from STMicroelectronics.

In one embodiment, sensor 602 may be positioned, for example, at the thorax of the patient or at the abdomen of the patient. In one embodiment, as shown in FIG. 5, the accelerometer is positioned at the lower ribs, roughly halfway between the central and lateral position. The positioning of the accelerometer shown in FIG. 5 allows monitoring of both the respiration characteristic and the heart rate, as well as the physical activity. In another embodiment, sensor 602 may be positioned such that the sensor is in close proximity with at least a portion of the patient's body. In one embodiment, the sensor 602 may be a part of a wearable band (that can be worn on the wrist, waist, arm or any other portion of the patient's body for example) or may be part of wearable garment worn by the patient.

Processor 606 is configured to 1) continuously receive acceleration data in at least the axes over a period of time, 2) determine the respiration rate data and the heart rate data from the accelerometer data, 3) determine physical activity data associated with each of the respiration rate data and the heart rate data, and 4) analyze the physical activity data, heart rate data and the respiration data to detect the onset of exacerbations in the patient based on the predetermined criteria.

In one embodiment, the predetermined criteria includes a comparison of a change in the respiration data and the heart rate data with a change in the physical activity data over the period of time. The change in the respiration data indicates an increase in the respiration rate, the change in the heart rate data indicates an increase in the heart rate, and the change in the physical activity data indicates a decrease in the physical activity.

In one embodiment, the period of time may be a course of a day. As noted above, the period of time may include a day, a week, a month, or any other desired time period. In one embodiment, an increase in the respiration rate and a decrease in the physical activity is determined as explained in system 200. In one embodiment, an increase in the heart rate is determined by comparing the patient's current heart rate to the patient's prior heart rate (e.g., a period of time ago) As noted above, the period of time may include a day, a week, a month, or any other desired time period.

In another embodiment, an increase in the heart rate is determined by comparing the patient's current heart rate to the patient's average heart rate. In one embodiment, the patient's average heart rate is determined by calculating an average or a median of the heart rate data taken over a past period of time.

In one embodiment, an increase in the heart rate is determined by comparing the patient's current heart rate to the baseline heart rate depending on the activity levels. As noted above, in one embodiment, the baseline heart rate may be measured for low, moderate and high activity levels to provide a reference.

In one embodiment, the respiration rate may be determined intermittently over period of time (i.e., the course of day) In one embodiment, the respiration rate is measured during rest and predetermined activity level (e.g., moderate walk for more than 2 minutes).

In one embodiment, a segmentation algorithm may be used to determine the respiration rate and the heart rate from the accelerometer data. The segmentation algorithm is configured to select the periods during which the respiration and heart rate may be determined.

In one embodiment, the segmentation of the data may be necessary because it may not always be possible to determine the respiration rate and/or the heart rate reliably during the physical activity using an accelerometer (and/or other sensors). In one embodiment, the segmentation algorithm serves to automatically identify the periods of time during which the respiration rate and/or the heart rate can be determined reliably. In one embodiment, because the respiration rate and/or the heart rate doesn't immediately return to baseline values after an activity this is not a problem for the method.

In one embodiment, about 20-30 seconds of good respiration rate data is sufficient to determine the respiration rate reliably. In one embodiment, about 20-30 seconds of good heart rate data is sufficient to determine the heart rate reliably.

In one embodiment, the physical activity associated with this respiration rate and/or this heart rate value may then be the averaged over the last 5 minutes or 15 minute period rather than just that 20-30 seconds during which the respiration rate and/or the heart rate was calculated. In one embodiment, the physical activity in a 15-minute period preceding the time instances at which the respiration rate and heart rate have been determined reliably.

In one embodiment, processor 606 may include a data storage unit or memory (not shown) that is constructed and arranged to store the physical activity data, the heart rate, and the respiration data over the period of time. The stored data may be used for further processing, for example, for trending, and/or display.

When the predetermined criteria is satisfied, processor 606 is configured to transmit a signal to alarm device 608 to generate the alarm indication. The alarm indication may be generated to indicate that the onset of the exacerbations is detected. Alarm device 608 is similar to the alarm device 208 (as shown in FIG. 2) or alarm devices 308 and 310 (as shown in FIG. 3), and hence will not be explained in detail here.

Besides predicting the onset of an exacerbation of a patient, system 600 may be used in other circumstances where the simultaneous assessment of the physical activity, the respiration rate and the heart rate may provide a better diagnosis of a patient's disease status, e.g. for asthma patients.

In one embodiment, only an activity monitor is used to anticipate an exacerbation by a decrease in activity levels over time. In such an embodiment, questionnaires are used to assess dyspnea. In other words, questionnaires are used in addition to the activity monitoring as both a decrease in activity levels (or a constant activity level) in combination with an increase in dysnea provides information about an onset of an exacerbation.

In one embodiment, only a respiration rate monitor only to anticipate an exacerbation by an increase in respiration rate over time. In one embodiment, trends in respiration rate are compared with the baseline respiration rate measurements to provide an indication of what constitutes as a significant increase in respiration rate and hence dyspnea. In such an embodiment, this increase should also remain relatively constant for a predetermined length of time.

In one embodiment, the acquired measurements (i.e., the physical activity data over the period of time, the heart rate data over a period of time and/or the respiration data over a period of time) may be used to calculate a single value, for example, an exacerbation risk score. The exacerbation risk score may be used in Early Warning Scoring Systems, for example, used by Rapid Response Teams. The exacerbation risk score may be used in the Early Warning Scoring Systems along with other known risk factors for deterioration, such as pulse rate, for example.

In one embodiment, systems 200, 300 and 600 may each include a single processor to detect the onset of the exacerbation based on predetermined criteria, wherein the predetermined criteria comprises a comparison of a change in the respiration data with a change in the physical activity data. In another embodiment, systems 200, 300 and 600 may each include multiple processors, where each processor is configured to perform a specific function or operation. In such an embodiment, the multiple processors may be configured to detect the onset of the exacerbation based on predetermined criteria, wherein the predetermined criteria comprises a comparison of a change in the respiration data with a change in the physical activity data.

In one embodiment, a system for predicting an onset of an exacerbation in a patient is provided. The system includes means for measuring physical activity of the patient over a period of time to gather physical activity data; means for measuring a respiration characteristic of the patient over the period of time to gather respiration data; and means for detecting the onset of the exacerbation based on predetermined criteria, wherein the predetermined criteria comprises a comparison of a change in the respiration data with a change in the physical activity data.

In one embodiment, a system for predicting an onset of an exacerbation in a patient is provided. The system includes means for measuring physical activity of the patient over a period of time to gather physical activity data; means for measuring a respiration characteristic of the patient over the period of time to gather respiration data; means for measuring a heart rate of the patient over the period of time to gather heart rate data; and means for detecting the onset of the exacerbation based on predetermined criteria, wherein the predetermined criteria comprises a comparison of a change in the respiration data and a change in the heart rate data with a change in the physical activity data.

Embodiments of the invention, the processor, for example, may be made in hardware, firmware, software, or various combinations thereof. The invention may also be implemented as instructions stored on a machine-readable medium, which may be read and executed using one or more processors. In one embodiment, the machine-readable medium may include various mechanisms for storing and/or transmitting information in a form that may be read by a machine (e.g., a computing device). For example, a machine-readable storage medium may include read only memory, random access memory, magnetic disk storage media, optical storage media, flash memory devices, and other media for storing information, and a machine-readable transmission media may include forms of propagated signals, including carrier waves, infrared signals, digital signals, and other media for transmitting information. While firmware, software, routines, or instructions may be described in the above disclosure in terms of specific exemplary aspects and embodiments performing certain actions, it will be apparent that such descriptions are merely for the sake of convenience and that such actions in fact result from computing devices, processing devices, processors, controllers, or other devices or machines executing the firmware, software, routines, or instructions.

Although the invention has been described in detail for the purpose of illustration, it is to be understood that such detail is solely for that purpose and that the invention is not limited to the disclosed embodiments, but, on the contrary, is intended to cover modifications and equivalent arrangements that are within the spirit and scope of the appended claims. In addition, it is to be understood that the present invention contemplates that, to the extent possible, one or more features of any embodiment may be combined with one or more features of any other embodiment.

1. A computer-implemented method for predicting an onset of an exacerbation in a COPD patient, the method comprising:
measuring physical activity of the patient over a period of time by gathering physical activity data;
measuring a respiration characteristic of the patient over the period of time by gathering respiration data; and
executing, on a computer processor, a computer program module to detect the onset of the exacerbation based on a COPD relationship score, the score being determined based on an estimation of change in physical activity versus change in a respiration characteristic.

2. (canceled)

3. The method of claim 1, wherein detection of the exacerbation is based on the COPD score indicating an increase in the respiration characteristic.

4. The method of claim 1, wherein detection of the exacerbation is based on the COPD score indicating a decrease in the physical activity.

5-8. (canceled)

9. A system for predicting an onset of an exacerbation in a COPD patient, the system comprising:
(a) a sensor configured to
(1) to measure physical activity of the patient over a period of time to gather physical activity data, and
(2) to measure a respiration characteristic of the patient over the period of time to gather respiration data; and
(b) a processor configured to detect the onset of the exacerbation based on a COPD relationship score, and wherein the processor is further configured to estimate the COPD relationship score from the change in physical activity versus the change in the respiration characteristic.

10. (canceled)

11. The system of claim 9, wherein the detection of the exacerbation is based on the COPD score indicating an increase in the respiration characteristic.

12. The system of claim 9, wherein the change in the physical activity data indicates a decrease in the physical activity.

13. (canceled)

14. (canceled)

15. The system of claim 9, wherein the respiration characteristic of the patient is a respiration rate or a respiration pattern.

16-30. (canceled)

31. A system for predicting an onset of an exacerbation in a patient, the system comprising:
means for measuring physical activity of the patient over a period of time to gather physical activity data;
means for measuring a respiration characteristic of the patient over the period of time to gather respiration data; and
means for detecting the onset of the exacerbation based a COPD relationship score, and wherein the means for detecting the onset of the exacerbation is further configured to estimate the COPD relationship score from the change in physical activity versus the change in the respiration characteristic.

32. (canceled)

33. The system of claim 31, wherein detection of the exacerbation is based on the COPD score indicating an increase in the respiration rate.

34. The system of claim 31, wherein the change in the physical activity data indicates a decrease in the physical activity.

35. (canceled)

36. (canceled)

37. The system of claim 31, wherein the respiration characteristic of the patient is a respiration rate or respiration pattern.

38-45. (canceled)

46. The method of claim 1, further comprising measuring a heart rate of the patient over the period of time; and wherein the detection of the onset of exacerbation is further based on a decrease in physical activity accompanied by an increase in heart rate.

47. The system of claim 5, wherein the processor is further configured: to measure a heart rate of the patient over the period of time; and wherein the detection of the onset of exacerbation is further based on a decrease in physical activity accompanied by an increase in heart rate.

48. The system of claim 9, further comprising means for measuring a heart rate of the patient over the period of time; and wherein the detection of the onset of exacerbation is further based on a decrease in physical activity accompanied by an increase in heart rate.
